(19) United States
(12) Patent Application Publication
(10) Pub. No.: US 2013/0266655 A1
(43) Pub. Date: Oct. 10, 2013

(54) PROCESS FOR PRODUCING A MARINE MINERAL CONCENTRATE MADE FROM LITHOTHAMNIUM SEAWEED, AND THE MARINE MINERAL CONCENTRATE OBTAINED THEREFROM

(71) Applicant: Carlos Moreira Mendes, Belo Horizonte (BR)

(72) Inventor: Carlos Moreira Mendes, Belo Horizonte (BR)

(73) Assignee: PHOSTHER ALGAMAR LTDA., Rio de Janeiro (BR)

(21) Appl. No.: 13/442,277

(22) Filed: Apr. 9, 2012

(51) Int. Cl. A61K 36/04; B02C 23/08; B02C 19/00; A61K 9/48; A61P 29/00

(52) U.S. Cl. 424/490; 424/195.17; 241/28; 241/24.11

(57) ABSTRACT

Various embodiments of the present invention refer to a new process for transforming Lithothamnium seaweeds and obtaining a marine mineral concentrate made from Lithothamnium seaweed having superior and differentiated properties and activities.

### FIELD OF THE INVENTION

The present invention addresses processing technologies for calcareous seaweeds belonging to the Lithothamnium sp. genus, in order to provide a new and improved process for transforming Lithothamnium seaweeds and obtaining a Lithothamnium seaweed product having superior and distinguished properties and activities.

### BACKGROUND OF THE INVENTION

Calcareous seaweeds are represented by marine seaweeds belonging to about 34 genus and approximately 500 species, depending on their regions of occurrence in seas and oceans. (Dias, G. T. M. “Granulados bioclásticos—algas calcárias”, Brazilian Journal of Geophysics, 18 (3):307-318, 2000).

Among them, there are coralline seaweeds belonging to the Lithothamnium sp. genus and Corallinaceae family, which are photosynthetic seaweeds that live on the surface of the sea bottom, on the outer sides of crusts, substrates and sedimentary deposits or banks of these seaweeds, other seaweeds and calcareous sands, lithoclastic sands and gravels, and other marine matter. These seaweeds are constantly renewed, provided that they come into contact with natural light. Lithothamnium seaweeds are reddish pink in color, with the loss of color or discoloration indicating a process leading to death. Calcium carbonate and magnesium precipitate in the Lithothamnium seaweeds in the form of calcite crystals, containing more than 20 oligo-elements in variable quantities, such as iron (Fe), manganese (Mn), boron (B), nickel (Ni), copper (Cu), zinc (Zn), molybdenum (Mo), selenium (Se) and strontium (Sr). (DIAS, 2000; Carlos, A. C. et al. “Uso da alga Lithothamnium calcareum como fonte alternativa de cálcio nas rações de frangos de corte” Ciénc. Agrotec., Lavras, v. 35, n. 4, p. 833-839, July-August 2011).

Applications for Lithothamnium seaweeds have been reported in the fields of agriculture, water treatment, cosmetics, food and medicine. The free forms of the seaweeds (e.g. rhodoliths, nodules and fragments, among others) are feasible for commercial explitation and industrial processing. Lithothamnium seaweeds may be used in their natural state, being extracted manually (e.g. by fishing nets, divers and others) or mechanically (e.g. mechanical dredges, suction vessels, suction machines and others), being run through conventional washing, dehydration and grinding processes (DIAS, 2000; CARLOS, 2011).

The main chemical and physical features that underpin the properties of these seaweeds are: (i) the availability of micronutrients that are adsorbed into the cell walls, thus being easily assimilable by plants and animals (these oligo-elements are essential at the physiological level, in basic biochemical reactions); and (ii) the high porosity of these seaweeds (>40%), which results in a larger specific surface for action (DIAS, 2000).

France is the world's largest producer of granules extracted from calcareous seaweeds. In Brittany, about 16 ports handle more than 600,000 tons a year of calcareous matter (mainly Lithothamnium seaweed). The total output of granules in France reaches about 1% of the nation's total output of granules in that Country. In 1998, the Union Nationale des Producteurs de Granulats (UNPG) clustered together more than 1,000 producing and handling companies, generating about 15,000 jobs and handling 346 million tons, with financial flows of 14 billion French Francs (more than 2 billion Euros) (DIAS, 2000).

Until 1985, the average annual volumes dredged from the North Atlantic, the North Sea and the Baltic Sea reached 1 million m³ of carbonaceous matter drawn from Lithothamnium seaweed (DIAS, 2000).

Brazil's continental shelf is the world's largest area covered by Lithothamnium seaweed. The potential for the commercial operation of these seaweed deposits is greater than that one of their French counterparts (DIAS, 2000).

Lithothamnium seaweeds are used as enriching agents or sources of calcium or natural/plant minerals in food products designed for human and animal consumption. Examples of products for human consumption include the Dessert au Chocolat plant-based chocolate-flavored dessert made by the Grandeur Nature company in France, the BioV line organic drink made by the Jasmine Alimentos company in Brazil, and the Litho Calcium natural food supplement made by the Biovita company in Brazil. Examples of animal products include chicken feed as assessed by CARLOS (2011), a mineral supplement for dogs as assessed by Costa Neto, J. M. et al. “Farinha de algas marinhas (“Lithothamnium calcareum”) como suplemento mineral na cicatrização óssea de autoenxerto cortical em cães.” Rev. Bras. de Saúde e Prod. An., v. 11, No 1, page 217-230, January-March 2010, a food supplement for ruminants as assessed in Orsine, G. F. et al. “Efeito da fonte de cálcio (Calcário vs Lithothamnium calcareum) na digestibilidade aparente do feno de capim Brachiaria decumbens Staph cv. Basiliski” Anais Esc. Agron. e Vet., 19 (1):49-58, January-December 1989, and the Superal® food supplement for animals made by the Algarea company in Brazil.

Lithothamnium seaweeds are also used as organic fertilizers, soil correctors and catalysts. Examples of commercial products are Algen made by the Oceana Brasil company in Brazil and Alfertil® made by the Algarea company in Brazil.

Patent application PCT/IB1998/00142, published on Aug. 6, 1998, in the name of Aquacal Limited (Ireland), refers to a product formation process using seaweeds belonging to the Lithothamnium genus, for the medical treatment of human beings and animals, wherein fragments and siliceous materials are removed from an excavated matter, said matter being washed thoroughly in seawater and freshwater (in order to lower the sodium content and to remove impurities), after which it is bleached, cleaned, sterilized with hydrogen peroxide, dried in a sterile fluidized bed, ground under controlled conditions and packed under aseptic conditions in capsules, tablets or emulsions.

Patent application DE 10 2006 056 454.5, published on May 29, 2008 in the name of Bluebiotech International GmbH (Germany), refers to a nutritional supplement comprising a blend of three seaweeds (Spirulina platensis, Chlorella pyrenoidosa and Lithothamnium calcerum), in the form of granules. The formation process for the supplement consists of (a) spray-drying the seaweed; (b) blowing the dry seaweed through a wet-process granulation device; (c) pulverizing sterile water in the device; (d) agglomerating the dry powder with water spray; (e) collecting the granules from the bottom of the device; and (f) selecting granules in compliance with the required granulometry, through sieving.

Patent application PCT/GB1999/03580, published on May 11, 2000 in the name of Aquacal Limited (Ireland), refers to food and cosmetic compositions containing calcareous matter derived from Lithothamnium seaweed. In order to obtain this seaweed, a process is performed, which consists of purifying, concentrating and encapsulating 0.5 grams of concentrate. The purification step consists of washing the seaweeds in seawater and freshwater, while also removing impurities. The matter is then separated and run through another intensive washing, with bleaching and sterilization by hydrogen peroxide for 8 to 24 hours, after which it is washed with water, dried in a sterile fluidized bed and ground under controlled conditions. The product is encapsulated in quantities of 0.5 grams.

A Brazilian company—Jose Roberto Faria Jacarei M E (www.lithothamnium.com.br)—makes granules and powders from Lithothamnium seaweed for agricultural, pharmaceutical, cosmetic and nutritional purposes. The material is collected from dead seaweed banks (discolored) and is transported by vessels. The seaweeds are dried and then processed to obtain granules or powders with a mean particle size of 5 μm to 60 mm.

A Brazilian company—Oceana Brasil (www.oceanabrasil.com.br)—makes granules, powders, brans and liquids from Lithothamnium seaweed, for agricultural purposes. It is extracted from the Tutóia region of Maranhão State, Brazil, using a Hopper-type dredge. The seaweeds are processed through drying, grinding, bagging and storing the product in an industrial structure. This process is free from any manual contact at all steps, and it is monitored by automated equipment.

A Brazilian company—Algarea S.A. (www.algarea.com.br)—makes the Alfertil soil fertilizer and the Superal food supplement for animals from Lithothamnium seaweed. The seaweed is extracted along the Espirito Santo State coastline, Brazil, from reserves consisting only of calcareous seaweeds sediments, with no quartz sand or shells. The extraction process uses a bucket dredge, which is mounted on a self-propelling vessel. The beneficiation process is handled at an industrial facility with a port at the site, which comprises the following steps: pre-grinding in a hammer mill, drying at controlled temperatures (in order to preserve the integrity of the seaweed components), final grinding in a roller mill, bagging in polyethylene bags by automatic machines, and storage of the end product. This industrial process is free from manual contact at all steps, and it is conducted and monitored by electronic equipments. FIG. 1 illustrates this process (legends: 1) extraction step; 2) self-propelling vessel with crane and bucket dredge; 3) storage yard; 4) drying step; 5) front end loader; 6) hopper; 7) conveyor belt; 8) mill; 9) rotating dryer; 10) exhaust/filter set; 11) transporting conveyor; 12) bucket loader; 13) storage silo; 14) grinding step; 15) additives dosing device; 16) roller mill; 17) cyclone; 18) sleeve filter; 19) exhaust fan; 20) bagging step; 21) bagging machine; 22) inventory; 23) loading).

The state of the art of the present invention consists of processes that encompass a simple succession of automated grinding and sieving steps, some of them making use of chemical reagents or substances. These processes use blended marine raw materials containing the seaweeds of interest mixed in with dead seaweeds, other seaweeds and assorted impurities, debris and marine sediments. The processes of the state of the art produce Lithothamnium seaweed products having poorer formulations, higher loss of content levels of their natural components, and less activities and fewer biological properties.

### BRIEF SUMMARY

The present invention refers to a process for producing a marine mineral concentrate made from Lithothamnium seaweed.

Another embodiment of this invention is a marine mineral concentrate made from Lithothamnium seaweed, obtained through the process of the invention.

The technical problem addressed in this application refers to a process for producing Lithothamnium seaweed particulates, whose final product conserves the same composition or the same properties as live Lithothamnium seaweeds in their natural habitat.

This invention solves the above-mentioned technical problem via:

1) a process for producing a marine mineral concentrate made from Lithothamnium seaweed, in which the above-mentioned process comprises a specific succession of more effective steps, reducing or avoiding losses of raw materials, enhancing the yield of the final product and obtaining a final product whose quality is unexpectedly superior, when compared to the products or processes of the state of the art; and

2) a product (marine mineral concentrate made from Lithothamnium seaweed) produced through the process encompassed by the invention, which maintains the same or approximately the same levels or original content levels of its components, providing greater bioavailability and better properties/activities, when compared to the products available in the state of the art.

The process addressed by the invention produces a product made from Lithothamnium seaweed having a richer constitution and little or no loss of content level in its natural components, and with greater activity and better biological properties. A detailed study by Soares, C. M. “Estudo quimico da alga Lithothamnion calcareum e avaliação da atividade inibitória do rolamento de leucócitos” School of Pharmacy, Federal University of Minas Gerais (UFMG) 2009, assessed the chemical composition of Lithothamnium calcareum red seaweed and its potential for anti-inflammatory activity, demonstrated through a leukocyte rolling inhibition model.

### BRIEF DESCRIPTION OF THE DRAWINGS

FIG. 1—FIG. 1 presents a schematic representation of the process for producing powders and granules made from Lithothamnium seaweed of the state of the prior art.

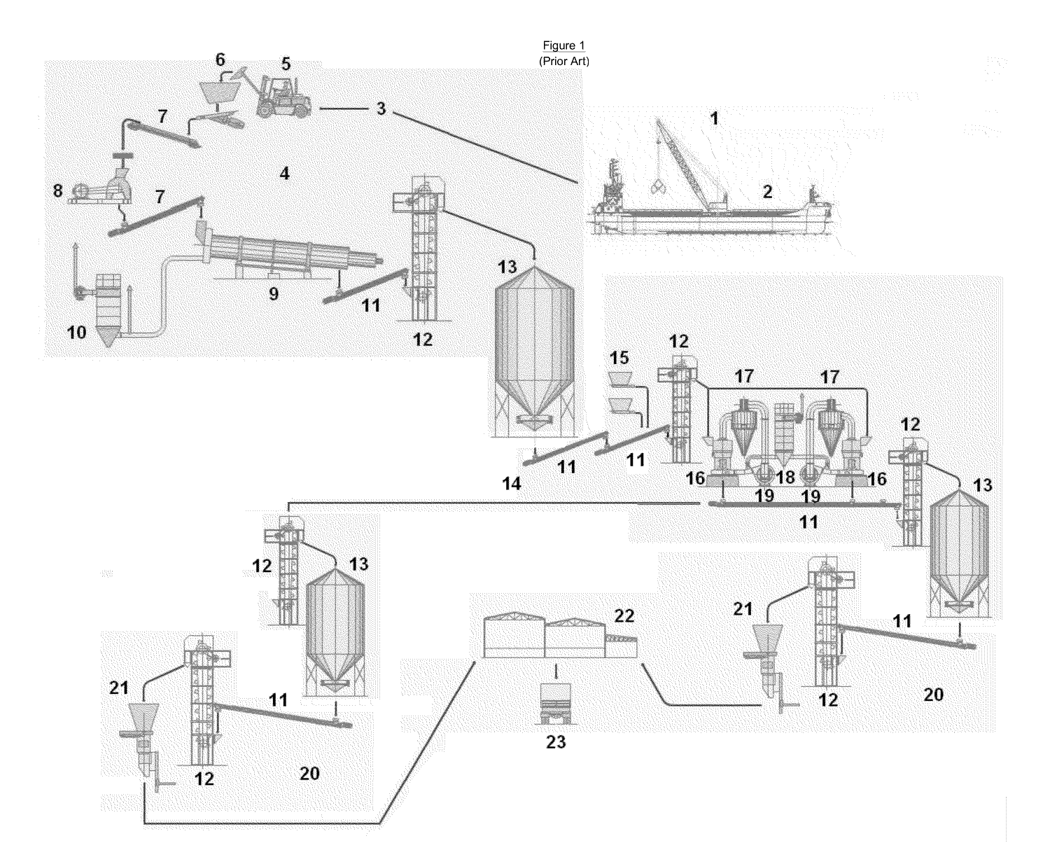

FIG. 2—FIG. 2 presents a schematic representation of a process for producing the marine mineral concentrate in accordance with various embodiments of the invention.

### DETAILED DESCRIPTION

This invention refers to a process for producing a marine mineral concentrate, which comprises:
collecting seaweeds belonging to the Lithothamnium sp genus;
at least one step of selection of the Lithothamnium seaweeds;
at least one step of reduction of the mean particle size of the Lithothamnium seaweeds; and
stabilization of the Lithothamnium seaweeds.

In various embodiments of the invention, the process for producing a marine mineral concentrate comprises:

(a) collection (1) and first selection (2) of seaweeds belonging to the Lithothamnium sp genus;

(b) second selection (3) and first reduction of the moisture content (4) of the Lithothamnium seaweeds;

(c) first reduction in the mean particle size (5) and third selection (6) of the Lithothamnium seaweeds;

(d) second reduction of the mean particle size (7) and fourth selection (8) of the Lithothamnium seaweeds;

(e) concentration (9) of the Lithothamnium seaweeds;

(f) stabilization (10) of the Lithothamnium seaweeds;

(g) second reduction of the moisture content (11) of the Lithothamnium seaweeds;

(h) third reduction of the moisture content (12) and granulometric classification (13) of the Lithothamnium seaweeds; and

(i) packaging (14) of the Lithothamnium seaweeds.

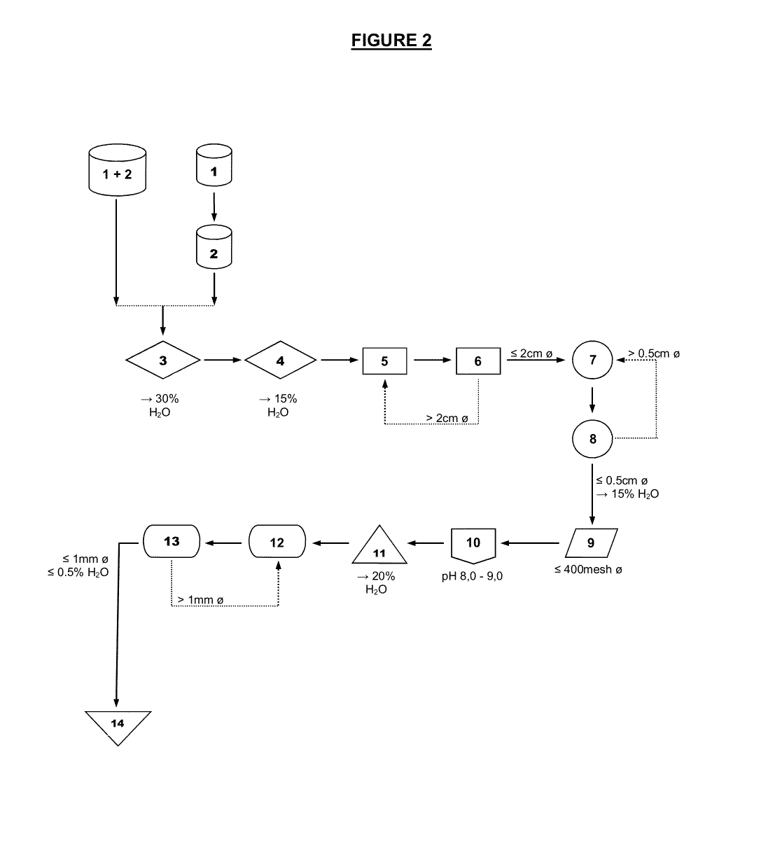

FIG. 2 illustrates this process according to various embodiments.

The process addressed by various embodiments of the invention comprises the following steps:

(a) Collection and First Selection of the Lithothamnium Seaweed:

The collection or extraction of seaweeds belonging to the Lithothamnium sp genus normally takes place in its natural habitat (seas and oceans). The collection site may not be located at or close to industrial facilities and sanitary or sewage networks.

Together with (concomitantly) or immediately after (sequentially) the above-mentioned collection, a first selection of the seaweeds takes place, through which the free forms of the Lithothamnium seaweeds are selected, whose coloring ranges from reddish to greenish, indicating live seaweeds. Discolored or white seaweeds are not useful for the purposes of the invention and are discarded, as they have already been dead for some time and are more crystallized, with heavier losses of their components and consequently their original properties and activities. Other seaweeds and calcareous sands, lithoclastic gravels and sands, shells, corals and other marine matter are also discarded. In certain embodiments, the above-mentioned free forms of the Lithothamnium seaweeds are in the form of rhodoliths, nodules and fragments, among others of this type.

The collection and the first selection are handled by conventional manual and/or mechanical techniques. In certain embodiments, the above-mentioned manual techniques comprise direct collection by hand or in fishing nets, and the above-mentioned mechanical techniques consists of suction or dredging by vessels commonly used in the technique. In other embodiments, the collection may be handled mechanically and the first selection may be handled manually, in order to obtain selected Lithothamnium seaweed matter of superior quality, substantially free from impurities and undesirable materials.

Once collected and selected, the Lithothamnium seaweeds are transported from the collection and/or selection area to the industrial facility, in a conventional manner, as for example transporting the seaweeds in plastic bags, barrels and casks.

(b) Second Selection and First Reduction in the Moisture Content of the Lithothamnium Seaweeds:

Step (b) and the other subsequent steps are conducted in an industrial environment.

The matter collected and selected initially during the previous step (a) is run through a second selection, in order to separate the material of interest from impurities and undesirable matter, whereby such material of interest consists predominantly of Lithothamnium seaweed. Discolored or white seaweeds, other seaweeds and calcareous sands, lithoclastic gravels and sands, shells and corals, as well as marine matter other than Lithothamnium seaweed are discarded. It is noted that the absence or poor conduct of the previous step (a) may result in the loss of material consisting of up to 40% of the total collected seaweeds.

The above-mentioned second selection of Lithothamnium seaweed is handled through conventional manual and/or mechanical techniques. Manual techniques may be employed, particularly with the application of water to the material resulting from the previous step (a), when it is also desalinized. In various embodiments, the above-mentioned application of water may take place on a table, bench or conveyor belt with a spray of water and/or in a rotating or spinning drum with running water.

The material resulting from the second selection, which consists predominantly of Lithothamnium seaweed, presents a moisture content of up to about 30%. This moisture content is measured or controlled through conventional methods in this technique, as for example measuring weight differences after drying in a kiln. This material is then run through the first reduction of the moisture content, in certain embodiments through conventional drying, and in other embodiments by centrifuging, until reaching a moisture content of about 15%. It is noted that a moisture content higher than about 15% may result in agglomeration due to excess water, with adverse effects on the subsequent step.

(c) First Reduction of the Mean Particle Size and Third Selection of the Lithothamnium Seaweeds:

In step (c), the Lithothamnium seaweed matter resulting from the previous step (b) undergoes its first reduction in the mean particle size, and a third selection.

According to various embodiments, the above-mentioned first reduction of the particle size takes place through conventional grinding techniques. In other embodiments, the above-mentioned grinding consists of crushing or grinding, particularly when said crushing/grinding being performed by jaw crushers, hammer mills or roller mills.

The Lithothamnium seaweed particulates run through a third selection, which is handled manually and/or mechanically. In certain embodiments, the above-mentioned third selection of Lithothamnium seaweed is performed manually.

The Lithothamnium seaweeds are selected with mean particle sizes smaller than about 2 cm. Said mean particle size is determined through regulating the grinding equipment. Particulate matter whose dimensions exceed 2 cm is returned to run through the first particle size reduction step again, and subsequently the repetition of the third selection, until the remaining Lithothamnium seaweed matter presents a mean particle size smaller than about 2 cm.

(d) Second Reduction in the Mean Particle Size and Fourth Selection of the Lithothamnium Seaweeds:

During step (d), the Lithothamnium seaweed matter resulting from the previous step (c) undergoes a second reduction of the mean particle size and a fourth selection.

According to various embodiments, the above-mentioned second reduction of the particle size takes place through conventional grinding techniques. In certain embodiments, the above-mentioned grinding consists of crushing or grinding, particularly said crushing/grinding being carried out by jaw crushers, hammer mills or roller mills.

The Lithothamnium seaweed particulates are run through a fourth selection, which is handled manually and/or mechanically. In certain embodiments, said fourth selection of Lithothamnium seaweed is handled manually.

Lithothamnium seaweeds are selected with a mean particle size smaller than about 0.5 cm. Said mean particle size is determined by regulating the grinding equipment. Particulates larger than 0.5 cm are fed back to repeat the second reduction of the particle size and subsequently a repetition of the fourth selection until the remaining Lithothamnium seaweed matter presents a mean particle size smaller than about 0.5 cm.

During step (d), the moisture content of the matter obtained from Lithothamnium seaweed particulates is controlled, which must be kept at about 15%. In certain embodiments, said moisture content is controlled through conventional ovens or kilns.

(e) Concentration of the Lithothamnium Seaweeds:

Step (e) of concentration of the material consisting of Lithothamnium seaweed particulates comprises the selective wet-process grinding thereof, through successive sub-steps. The material is concentrated through granulometric control, distinguishing the particulate matter by hardness and dimension.

Sub-step (e-1) comprises the primary grinding of the material, during which a third reduction in the mean particle size of the material takes place. This sub-step is, according to various embodiments, handled over a briefer period of time, and in certain embodiments such is handled in about 1 to 2 hours. The mill may according to various embodiments be a conventional mill, and in certain embodiments, such may be a rotating ball mill.

Sub-step (e-2) comprises the primary sieving of the material, through which the harder/coarser particulate matter is separated out, and the finer particulate matter is selected. The above-mentioned sieving is in certain embodiments conducted through a conventional 100 mesh sieve. Particulate matter trapped in the mesh (i.e. having a mean particle size larger than 100 mesh) is discarded and the matter screened through this mesh is returned to the process in the subsequent step.

Sub-step (e-3) comprises the secondary grinding, during which a fourth reduction takes place of the mean particle size of the material. This sub-step is in certain embodiments conducted over a longer period of time, which may in at least one embodiment be over about 2 to 4 hours. The mill may be according to various embodiments a conventional mill, which may in certain embodiments be a rotating ball mill. It is noted that the length of time required to conduct sub-step (e-3) must be approximately twice the length of time for conducting sub-step (e-1).

Sub-step (e-4) comprises the secondary screening, during which the harder/coarser particulate matter is separated and the finer particulate matter is selected. The above-mentioned screening is according to various embodiments conducted through a conventional 400 mesh screen. Particulate matter trapped in the mesh (i.e. having a mean particle size above 400 mesh) with particle sizes between 100 and 400 mesh may be used as a by-product for other applications, as for example facial defoliants. The material passing through the 400 mesh screen continues for processing in the next step. The secondary screening (sub-step (e-4)) is according to various embodiments conducted with the application of water, in order to ease the passage of very fine particles.

(f) Stabilization of the Lithothamnium Seaweeds:

The Lithothamnium seaweed particles obtained during the previous step (e) are subjected to pH measurements, and are then left in repose to decant, when the supernatant liquid separates from the matter obtained from the Lithothamnium seaweed particles, which form a sediment on the bottom. According to various embodiments, said repose should last from around fifteen days to three months, depending on the temperature conditions (the tighter the temperature control of the environment, the shorter the duration of the repose—room temperature is the optimum temperature for the process). In certain embodiments, said repose takes place in a room, a chamber or an area at room temperature, while in still other embodiments, such may occur in a climatized room or chamber.

During step (f), the pH is controlled for the matter obtained from Lithothamnium seaweed particulates in repose. The pH of said matter obtained during the previous step (e) is measured at the beginning of step (f), varying from about 8.0 to about 9.0. During the repose period with the decantation of the material, its pH is measured constantly or regularly. There is a reduction of the initial pH, reaching an intermediate pH of about 7.0. The Lithothamnium seaweed particulates left in repose and decanted are removed during step (f), provided that its final pH is about 8.0 to about 9.0. Alterations to the pH that occur during the stabilization of the material entering step (f) and the material leaving step (f) are caused by the biological activity of the Lithothamnium seaweeds under said repose and decantation conditions.

The Lithothamnium seaweed matter obtained during step (f) has the appearance of a “pulp”, and already presents the chemical properties and biological activities of the final product.

(g) Second Reduction of the Moisture Content of the Lithothamnium Seaweeds:

The Lithothamnium seaweed matter obtained during the previous step (f) (or “pulp”) is run through a second reduction of its moisture content, which may in certain embodiments be through filtration. Said filtration is conducted in compliance with any conventional filtration technique. Particularly, said filtration is handled through a vacuum. The pulp is filtered until its moisture content does not exceed about 20%.

(h) Third Reduction in the Moisture Content and Granulometric Classification of the Lithothamnium Seaweeds:

The Lithothamnium seaweed matter obtained during the previous step (g) (or “cake”) is run through a third reduction in its moisture content, in certain embodiments through drying, in order to result in its fragmentation. Said drying is conducted in compliance with any conventional drying technique. Particularly, said drying takes place in laboratory kilns and industrial ovens, and even more particularly with control over the moisture content, time and temperature. The moisture content of the cake (filtered pulp) must range between 8% and 12%. The temperature must never exceed 60° C., and the time required is as long as needed for the moisture content to reach a value close to that previously established, which is obtained by testing the filtered matter during the subsequent step that reduces the particle size.

The dried and fragmented Lithothamnium seaweed matter is classified granulometrically by running it through conventional granulators of the state of the art. The granulation mesh should according to various embodiments be up to 1 mm. Said granulators should in those and still other embodiments be screens of up to 1 mm. The Lithothamnium seaweed particulates trapped in the granulators are returned to said drying step that precedes the granulometric classification for further fragmentation and subsequently further granulometric classification in granulators, until all the matter runs through the granulators reaching a mean particle size of up to 1 mm.

The Lithothamnium seaweed particles (up to 1 mm) is then run through a new drying step, in order to attain a constant weight and moisture content of up to about 0.5%. Said drying is conducted in compliance with any conventional drying technique. Particularly, said drying is handled through laboratory kilns and industrial ovens, and even more particularly controlling the moisture content of up to about 0.5% with a temperature of up to about 60° C.

A final Lithothamnium seaweed granulate is obtained of up to 1 mm, also called “marine mineral concentrate”. According to various embodiments, said granulate is cycled through a new drying and granulometric classification route, similar to that one described in step (h) above, in order to reduce its mean particle size even more, until obtaining powders up to 100 mesh. The Lithothamnium seaweed powders obtained through the process addressed by the invention are also called “marine mineral concentrates”.

(i) Packaging the Lithothamnium Seaweeds:

The marine mineral concentrate obtained through the invention is packed in compliance with the packaging materials and techniques available in the industry. Particularly, marine mineral concentrate consisting of Lithothamnium seaweed powder obtained during step (h) above is packed in plastic bags and conventional packs. Even more particularly, the marine mineral concentrate consisting of Lithothamnium seaweed powder obtained during the previous step (h) is encapsulated in compliance with conventional encapsulation materials and techniques. The above-mentioned marine mineral concentrate of Lithothamnium seaweed may also be subject to conventional sterilization techniques, such as gamma ray sterilization, for example.

### EXAMPLES

### Example 1

### Process for Producing Marine Mineral Concentrate

Eighteen tons of marine matter containing Lithothamnium seaweed were collected manually by dive fishermen at two different points along the Espirito Santo State coastline in Brazil, at depths varying from ten meters to fifteen meters, and were transported in a fishing smack (traineira).

The collected Lithothamnium seaweed matter was transported in plastic bags to the industrial plant in Minas Gerais State, Brazil, where the Lithothamnium seaweeds were selected visually and manually on a steel bench, removing contaminants and dead seaweeds, and keeping only the live Lithothamnium seaweeds (visual selection by type and coloring). The above-mentioned selection was conducted while spraying fresh water on the seaweed matter spread out on the bench. The selected matter (Lithothamnium seaweeds) was dried by centrifuging and weighed, in order to reach a moisture content of approximately 15%. The weight obtained was 11 tons, representing a loss of about 40% of the initial material.

After visual selection, the Lithothamnium seaweed matter was sent to a centrifuge, in order to adapt the moisture content as required for reducing the particle size by crushing in a jaw crusher (conventional, especially adapted). The crusher was regulated in a manner whereby the manually selected material was reduced to particles of approximately 10 cm. After a further manual selection, this procedure was repeated, with the crusher being regulated to produce particles with a mean size of up 2 cm.

The Lithothamnium seaweed particulates were sent to a rotating ball mill (conventional, specially adapted) for grinding during around two hours, and was subsequently screened at 100 mesh. The matter trapped in the screen was sent back to the mill for grinding during a further three hours, with matter above 100 mesh then discarded and all matter below 100 mesh resulting from the two screening processes run through a 400 mesh screen. The matter between 100 and 400 mesh became a by-product, and the matter running through the 400 mesh was sent to the stabilization step. It was packed in casks and stored in a climatized room, where the stabilization occurred. Initially presenting a pH of approximately 7, the pH value tended to stabilize in a range between 8 and 8.5, based on the storage time and temperature. In other words, stabilization did not occur as a function of time or temperature, despite the importance of these factors in the production process, but rather the absence of variation in the pH.

The pulp obtained after decantation was vacuum filtered through a filter array, presenting a residual moisture content of approximately 15%, resulting a cake that subsequently ran through a primary drying step, in order to reduce its initial moisture content, in a kiln kept at 60° C., until reaching a moisture content of between 10% to 12%, for subsequent fragmentation and running through 2 mm screens. The particulate matter running through the screen returned to the drying step, until its moisture content fell below 8%, after which it was fragmented again, until all the processed matter presented mean particle sizes of less than 1 mm. This particulate matter was sent to the kiln at up to 60° C., with its final moisture content not exceeding 0.5%. A final Lithothamnium seaweed granulate was obtained measuring less than 1 mm, which was then submitted to gamma radiation for sterilization control.

### Example 2

### Process for Producing Marine Mineral Concentrate

Four tons of marine matter containing Lithothamnium seaweed were collected manually by fishermen under close supervision, at a specific point along the Espirito Santo State coastline, Brazil, at depths varying from 1.5 meters at low tide, with a manual first selection step being performed simultaneously with collection. Selection procedures based on coloring were adopted in order to determine the percentage of live Lithothamnium seaweed; selection through shape of the Lithothamnium seaweeds was used to determine the chemical elements; and selection by size of the Lithothamnium seaweeds was to ensure a lower silica (SiO₂) content, as well as encrustations such as shells etc. Lithothamnium seaweed matter with the predominant presence of rhodoliths consisting of reddish and greenish Lithothamnium seaweed was selected and obtained.

The selected Lithothamnium seaweed matter was transported in plastic bags to the industrial plant in Minas Gerais State, Brazil, where a second selection of the Lithothamnium seaweeds took place, conducted visually and manually on a steel bench, removing contaminants and dead seaweeds, while retaining only the live Lithothamnium seaweeds (visual selection by type and coloring). The second selection was conducted while spraying fresh water on the seaweed matter spread out on the bench. The matter resulting from the second selection (only the Lithothamnium seaweeds) was dried by centrifuging and weighed, in order to present a moisture content of approximately 15%. The weight obtained was 3.6 tons, representing a loss of the initial material at about 10%.

After visual selection, the Lithothamnium seaweed matter was sent to a centrifuge, in order to adapt its moisture content as required for reducing the particle size in a jaw crusher through crushing (conventional, especially adapted). The crusher was regulated in a manner whereby the selected manually matter was reduced to particles of up to approximately 10 cm. After a further manual selection, this procedure was repeated, with the crusher being regulated again for particles with a mean size of up to 2 cm.

The Lithothamnium seaweed particulates were sent to a rotating ball mill for grinding (conventional, especially adapted) during some two hours, and were subsequently screened at 100 mesh. The matter trapped on the screen was returned to the mill for grinding during a further three hours, with matter above 100 mesh discarded, and all matter below 100 mesh resulting from the two classification processes being run through a 400 mesh screen. The matter between 100 and 400 mesh became a by-product, while the matter running through at 400 mesh was sent to the stabilization step. It was packed in casks that were stored in the climatized room, where stabilization occurred, initially presenting a pH of approximately 7. The pH value tended to stabilize at a range between 8 and 8.5, as a function of the storage time and temperature. In other words, stabilization did not occur as a function of time or temperature, despite the importance of these factors in the production process, but rather the absence of variability in the pH.

The pulp obtained after decantation was vacuum filtered in a filter array, presenting a residual moisture content of approximately 15%, and resulting in a cake that subsequently ran through a primary drying process, in order to reduce its initial moisture content in a kiln at 60° C., until reaching a moisture content of between 10% to 12%, for subsequent fragmentation and running through two 2 mm screens. The particulate matter running through the screen returned to the drying process, until its moisture content fell below 8%, when it was fragmented once again, until all the matter submitted presented mean particle sizes of less than 1 mm. This particulate matter was sent to the kiln at up to 60° C., whereby its final moisture content did not exceed 0.5%. A final Lithothamnium seaweed granulate was obtained of less than 1 mm, which was submitted to gamma radiation for sterilization control.

1. A process for producing a marine mineral concentrate, said process comprising:
collecting seaweeds belonging to the Lithothamnium sp genus;
at least one step of selection of the Lithothamnium seaweeds;
at least one step of reduction of the mean particle size of the Lithothamnium seaweeds; and
stabilization of the Lithothamnium seaweeds.

2. The process according to claim 1, wherein said at least one selection step of the Lithothamnium seaweeds comprises the step (a) of first selection of the Lithothamnium seaweeds.

3. The process according to claim 2, wherein said collection of the Lithothamnium seaweeds and the step (a) of first selection of the Lithothamnium seaweeds are conducted sequentially or concomitantly.

4. The process according to claim 2, wherein, in step (a), the first selection of the Lithothamnium seaweeds selects its free forms from the group consisting of rhodoliths, nodules and fragments.

5. The process according to claim 2, wherein the collection of the Lithothamnium seaweeds and the step (a) of first selection of the Lithothamnium seaweeds are handled at least one of manually and mechanically.

6. The process according to claim 5, wherein the collection is handled mechanically and the first selection is performed manually, in order to obtain selected Lithothamnium seaweed matter of superior quality, substantially free from impurities and undesirable materials.

7. The process according to claim 1, wherein said at least one selection step of the Lithothamnium seaweeds comprises the step (b) of second selection of the Lithothamnium seaweeds.

8. The process according to claim 7, wherein, in step (b), the second selection of the Lithothamnium seaweeds is performed at least one of manually and mechanically, in order to obtain selected Lithothamnium seaweed matter of superior quality, substantially free from impurities and undesirable materials.

9. The process according to claim 8, wherein the second selection is performed manually.

10. The process according to claim 8, wherein water is applied to the Lithothamnium seaweed matter, when it is also desalinized.

11. The process according to claim 8, wherein, in step (b), the selected Lithothamnium seaweed matter is additionally subjected to a first reduction of its moisture content, in order to reach a moisture content of up to about 15%.

12. The process according to claim 1, wherein said at least one reduction step of the mean particle size of the Lithothamnium seaweeds comprises the step (c) of first reduction of the particle size.

13. The process according to claim 12, wherein, in step (c), said first reduction of the particle size takes place through at least one of crushing and grinding, selecting Lithothamnium seaweed particles with a mean particle size smaller than about 2 cm.

14. The process according to claim 1, wherein said at least one reduction step of the mean particle size of the Lithothamnium seaweeds comprises the step (d) of second reduction of the particle size.

15. The process according to claim 14, wherein, in step (d), said second reduction of the particle size takes place through at least one of crushing and grinding, selecting Lithothamnium seaweed particles with a mean particle size smaller than about 0.5 cm.

16. The process according to claim 14, wherein said moisture content of the matter obtained from Lithothamnium seaweed particles is up to about 15%.

17. The process according to claim 1, wherein said at least one reduction step of the mean particle size of the Lithothamnium seaweeds comprises the step (e) of concentration of the matter obtained from Lithothamnium seaweed particles.

18. The process according to claim 17, wherein, in step (e), the concentration of the matter obtained from Lithothamnium seaweed particulates comprises the selective wet grinding thereof.

19. The process according to claim 17, wherein said concentration comprises sub-step (e-1), wherein it is conducted a primary grinding of the matter obtained from Lithothamnium seaweed, a third reduction occurring in its mean particle size.

20. The process according to claim 17, wherein said concentration comprises sub-step (e-2), wherein it is carried out a primary sieving of the matter obtained from Lithothamnium seaweed, a selection of the particulate matter occurring with a mean particle size of less than 100 mesh.

21. The process according to claim 17, wherein said concentration comprises sub-step (e-3), wherein it is conducted a secondary grinding of the matter obtained from Lithothamnium seaweed, a fourth reduction occurring in its mean particle size.

22. The process according to claim 17, wherein said concentration comprises sub-step (e-4), wherein it is carried out a secondary sieving of the matter obtained from Lithothamnium seaweed, selection of the particulate matter occurring with a mean particle size of less than 400 mesh.

23. The process according to claim 22, wherein water is applied during the secondary sieving.

24. The process according to claim 19, wherein sub-steps (e-1) to (e-4) are performed successively.

25. The process according to claim 19, wherein sub-step (e-3) lasts approximately twice as long as the time required to perform sub-step (e-1).

26. The process according to claim 1, wherein said stabilization of the Lithothamnium seaweeds comprises the step (f) of stabilization of the Lithothamnium seaweeds.

27. The process according to claim 26, wherein, in step (f), the Lithothamnium seaweed matter is left in repose for decantation, preferably for a period of time lasting from about 15 days to about 3 months, depending on temperature conditions.

28. The process according to claim 26, wherein the pH of the matter obtained from Lithothamnium seaweed varies in the range of about 7.0 to about 9.0.

29. The process according to claim 28, wherein the pH of the matter obtained from Lithothamnium seaweed varies in the range of about 8.0 to about 9.0.

30. The process according to claim 1, said process further comprising the step (g) of second reduction of the moisture content of the matter obtained from Lithothamnium seaweed, whereby its moisture content does not exceed about 20%.

31. The process according to claim 1, said process further comprising the step (h) of third reduction of the moisture content of the matter obtained from Lithothamnium seaweed, whereby its moisture content does not exceed about 12%, and it is then classified granulometrically in a granulator with granulation meshes of up to 1 mm.

32. The process according to claim 1, wherein the Lithothamnium seaweed matter presents a moisture content of up to about 0.5%.

33. The process according to claim 1, said process further comprising the steps of:
(a) collection and first selection of seaweeds belonging to the Lithothamnium sp genus;
(b) second selection and first reduction of the moisture content of the Lithothamnium seaweeds;
(c) first reduction of the mean particle size and third selection of the Lithothamnium seaweeds;
(d) second reduction of the mean particle size and fourth selection of the Lithothamnium seaweeds;
(e) concentration of the Lithothamnium seaweeds;
(f) stabilization of the Lithothamnium seaweeds;
(g) second reduction of the moisture content of the Lithothamnium seaweeds; and
(h) third reduction of the moisture content and granulometric classification of the Lithothamnium seaweeds.

34. The process according to claim 1, wherein the final product made from Lithothamnium seaweed is a marine mineral concentrate in a form selected from the group consisting of brans, granules, powders or mixtures thereof.

35. The process according to claim 34, wherein the marine mineral concentrate is encapsulated in physiologically acceptable capsules.

36. Marine mineral concentrate, wherein said concentrate is obtainable from the process of claim 1.
